(19) United States
(12) Patent Application Publication
(10) Pub. No.: US 2024/0140443 A1
(43) Pub. Date: May 2, 2024

(54) DEVICE AND METHOD FOR ASCERTAINING THE ATTENTIVENESS OF A DRIVER OF A VEHICLE

(71) Applicant: ZF Friedrichshafen AG, Friedrichshafen (DE)

(72) Inventors: Julian King, Rankweil (AT); Rene Seidemann, Friedrichshafen (DE)

(73) Assignee: ZF Friedrichshafen AG, Friedrichshafen (DE)

(21) Appl. No.: 18/546,914

(22) PCT Filed: Nov. 30, 2021
(86) PCT No.: PCT/EP2021/083623

§ 371 Date: Aug. 17, 2023

(30) Foreign Application Priority Data
Feb. 17, 2021 (DE) 10 2021 201 498.4

(51) Int. Cl. B60W 40/08; A61B 5/00; A61B 5/0205; A61B 5/08; A61B 5/145; A61B 5/16; A61B 5/18; B60W 50/14

(52) U.S. Cl. CPC B60W 40/08; A61B 5/0077; A61B 5/0205; A61B 5/082; A61B 5/163; A61B 5/18; A61B 5/6893; A61B 5/7267; B60W 50/14; A61B 5/14546; B60W 2040/0827; B60W 2540/225; B60W 2540/229

(57) ABSTRACT

A device for determining driver alertness includes a gas sensor for continuously monitoring a driver's exhalations and/or volatile organic compounds emitted from the skin, and an analyzer for extracting at least one volatile biomarker in the exhalations and/or the emitted volatile organic compounds, wherein the biomarker is isoprene, and wherein the analyzer is configured to determine the isoprene concentration over the course of a predefined time period to obtain an isoprene concentration profile, determine peaks in the isoprene concentration profile, such that the alertness of the driver can be determined on the basis of the number of peaks within the predefined time period, and/or determine the driver's alertness on the basis of a frequency analysis of isoprene concentrations within the predefined time period, and/or determine the driver's alertness on the basis of random distributions of isoprene concentrations within the predefined time period.

The invention relates to a device for determining a vehicle driver's alertness, which has at least one gas sensor with which at least a portion of the exhalation and/or volatile organic compounds emitted from the driver's skin are monitored continuously, and an analyzer for extracting at least one volatile biomarker in the exhalations and/or the emitted volatile organic compounds. The invention also relates to a method for determining a driver's alertness.

Driver monitoring is becoming increasingly more important in assisted and automated driving up to SAE level 3. Early detection of fatigue and increased stress is of primary importance, along with continuous monitoring of the driver's alertness in the case of automated driving at SAE level 3. Drivers often make minor steering errors when they are less alert (tired), and attempt to correct them abruptly. This can be detected electronically by a steering angle sensor, which is frequently part of the ESP anti-skid system. A lane keeping assist can also be used to detect fatigue. These systems for detecting when a driver is insufficiently alert make use of an interpretation of the driver's behavior, e.g. when they are not remaining within lane markings.

EP 1 392 149 B1 discloses a method and system for determining the alertness of a vehicle driver in which at least one first movement property of at least a first part of a vehicle and at least one second movement property of at least a second part of the vehicle are detected, and at least one temporal relationship between the at least one first movement property and the at least one second movement property is used to determine and distinguish between which movements are caused by the driver and which movements are not caused by the driver, such that the alertness of the vehicle driver can be determined on the basis of the relationship between the movements caused by the driver and the movements not caused by the driver.

The object of the invention is to create a means with which traffic safety is further improved by determining a driver's alertness.

This problem is solved by a device that has the features of claim 1. It is also solved by a method that has the features of claim 12.

Advantageous designs, which can be used in and of themselves or in combinations with one another, can be derived from the dependent claims and the description.

The problem addressed by the invention is solved by a device for determining a vehicle driver's alertness, which contains
at least one gas sensor with which at least a portion of the exhalation and/or the volatile organic compounds emitted from a driver's skin are monitored continuously, and
an analyzer for extracting at least one volatile biomarker from the exhalation and/or the emitted volatile organic compounds,
wherein the biomarker is isoprene (2-methyl-1,3-butadiene), and wherein the analyzer is configured to determine the concentration of isoprene over the course of a predefined time period to obtain an isoprene concentration profile, and the analyzer is also configured to detect peaks in the isoprene concentration profile in order to determine the level of alertness on the basis of the number of peaks within the predefined time period, and/or the analyzer is also configured to determine the alertness on the basis of a frequency analysis of isoprene concentrations within the predefined time period, and/or the analyzer is also configured to determine the alertness on the basis of a random distribution (scattering) of isoprene concentrations within the predefined time period.

Human exhalations contain many hundreds of volatile organic compounds (VOCs, trace gases), the concentrations of which correspond to normal and pathological metabolic processes and physiological processes in the body. The same applies to trace gases emitted from human skin. It is acknowledged in the scope of the invention that the enormous progress in chemical analysis and the associated gas sensors make it possible to detect quantities of these substances in concentrations of only a few ppt (parts per trillion=1 in 1012 particles). Extremely small sensors can be used for this (<1 cm3), e.g. formed using metal oxide semiconductors.

The concentration of the biomarker isoprene (2-methyl-1,3-butadiene) is analyzed as a preliminary indicator of a lack of concentration and/or fatigue on the part of the driver. It has been established that this concentration is very inconsistent in the breath and emissions from the skin during the waking phases, i.e. there are numerous peaks at irregular intervals. This is because isoprene is flushed out of the muscles, even during subconscious contractions of the skeletal muscles, while the isoprene concentration remains constant during a sleeping phase or when a person is fatigued.

It has also been established that even in short time periods, a momentary nodding off (microsleep) or fatigue phase can be reliably detected from the characteristic irregular isoprene concentrations, i.e. from the typical and frequent concentration peaks during the waking phase.

The gas sensor system, or gas sensors, can comprise sensors for specific substances, as well as non-specific sensor arrays that react to changes in the overall composition of the gas volume.

Peak values can be determined changes in the isoprene concentration that exceed a predefined threshold value. The number of peaks within a predefined time period can then be used as a measure of a driver's alertness. If there are four peaks, for example, it can be assumed that the driver is very alert. If there is just an average number of peaks, this can be regarded as an indication of the onset of fatigue.

Alertness can also be determined from a random distribution (scattering) of isoprene concentrations within the predefined time period. Inactivity (fatigue/alertness) on the part of the driver can be detected on the basis of the random distribution (scattering) of the isoprene concentrations within the predefined time period.

The analyzer can also be configured to determine alertness on the basis of a frequency analysis of isoprene concentrations within the predefined time period.

This kind of distribution/frequency analysis is advantageous because isoprene concentrations change over the course of the day. Different foods can also affect the absolute isoprene concentration. Inactivity can be determined independently of the time of day and independently of individual drivers with a distribution/frequency analysis.

Alertness can also be determined on the basis of specific peaks and the frequency analysis, as well as the random distribution.

The device according to the invention is a non-invasive, contact-free and permanently available means for monitoring a driver to determine the driver's current level of alertness.

This determination can take place continuously while driving. This forms a substantial contribution to increasing traffic safety.

The at least one gas sensor and the analyzer are preferably designed to measure and evaluate the changes in isoprene concentration in realtime, e.g. triggered by breathing. This means that fatigue and microsleeping can be detected quickly.

The driver is preferably monitored continuously over the course of numerous successive time periods without breaks therebetween.

Another preferred embodiment contains a driver identification sensor. The analyzer is also configured to adjust the time period, and/or the determination of the alertness, and/or driver identification to the driver. This means that the invention can be tailored to a current driver, thus improving the quality of the determination of a driver's alertness.

The analyzer is preferably configured to adjust the time period, and/or determination of alertness, and/or driver identification with a machine learning process. This machine learning process can take place in an artificial neural network, using a classification/clustering process, regression process, decision tree learning/random forest classifier, or a reinforcement learning process.

This takes into account the fact that the isoprene concentration in an exhalation or emitted through the skin may differ significantly for each individual. This depends largely on the level of physical activity. It can also depend heavily on the driver's health. For this reason, absolute as well as relative and binary (presence or absence of trace gases) changes in the respective isoprene concentrations in comparison with an output or nominal concentration for a specific driver can be incorporated in the determination of the driver's alertness.

The device can also contain an additional sensor for detecting a driver's alertness. The device can also be configured to combine the alertness detected with this additional sensor and the alertness determined through the isoprene concentration to obtain an overall alertness rating.

This combination results in a more precise and/or quicker determination of the driver's alertness.

By way of example, the additional sensor can be an interior camera for detecting a driver's eyelid movements and/or pupil dilation, and/or head position and/or yawning frequency. The additional sensor can also be used to monitor pulse rates and/or breathing frequencies. There can also be numerous additional sensors.

In another embodiment, the at least one gas sensor is placed in the headrest on the driver's seat, and/or the driver's seatbelt, and/or the steering wheel, and/or the back of the driver's seat, and/or in the ceiling above the driver's seat.

By placing the at least one gas sensor near the mouth and skin of the driver, the isoprene concentration detection can be targeted on the driver. Interference from isoprene concentrations in other vehicle occupants is largely suppressed in this manner, in that they are not detected. The gas sensor can also be installed in air vents near the driver.

The device can also be designed to generate a visible, and/or tactile, and/or audible signal, if a driver's alertness falls below a first predefined level. An audible signal can be a warning sound, and a visible signal can be a blinking light. Tactile signals can comprise vibrations in the seat or steering wheel.

This further increases a driver's alertness and improves traffic safety.

The device can also be designed to generate control signals for evaluation in advanced driver assistance systems if the driver's alertness falls below a second predefined level. This level could result in life-threatening situations. In this case, control commands can be sent directly or indirectly, e.g. via a central vehicle control unit, to advanced driver assistance systems, e.g. a lane keeping assist, emergency assistance system, or emergency response system in the vehicle with which further necessary measures are then initiated. This can involve emergency braking or placing an eCall, for example.

There is an additional gas sensor in another embodiment, with which other volatile organic compounds emitted in exhalations and/or from the driver's skin are continuously detected. This can be obtained by placing the two gas sensors in different locations (steering wheel/seatbelt). The two gas sensors can also be identical.

This allows for a correction or confirmation of the isoprene concentration detected by the first or second gas sensor.

In another embodiment, the analyzer is designed to extract another volatile biomarker and to determine the isoprene concentration and the other volatile biomarker over the timer period to obtain an overall concentration profile and to determine the driver's alertness on the basis thereof.

The analyzer can continuously analyze the wakefulness and/or alertness of the driver using these biomarkers. Alertness can be more precisely determined using a combination of various biomarkers. In particular, the second biomarker can be a CO2 (carbon dioxide) level that can also indicate fatigue, because CO2 levels increase during sleep phases.

The object of the invention is also achieved with a method for determining a driver's alertness, which is carried out on a device such as that described above, comprising the following steps:
continuous detection of at least a portion of a driver's exhalations and/or the volatile organic compounds emitted from a driver's skin using a gas sensor,
extracting at least one volatile biomarker in the exhalation and/or the emitted volatile organic compounds by the analyzer, wherein this biomarker is isoprene (2-methyl-1,3-butadiene),
determining the isoprene concentration over a predefined time period to obtain an isoprene concentration profile,
determining peaks in the isoprene concentration profile to determine the driver's alertness on the basis of the number of peaks, and/or to determine the driver's alertness on the basis of a frequency analysis of isoprene concentrations within the predefined time period, and/or to determine the driver's alertness on the basis of random distributions (scatterings) of isoprene concentrations within the predefined time period.

The advantages of the device can also be obtained with the method.

Further properties and advantages of the present invention can be derived from the following description in reference to the drawings. Therein:

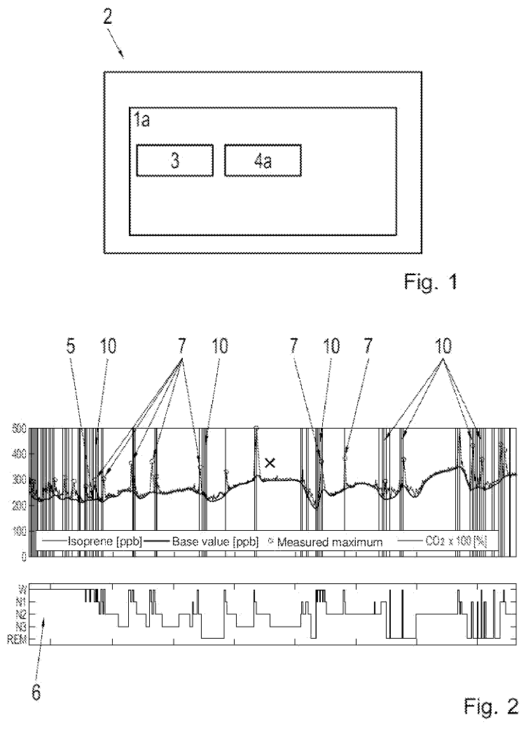

FIG. 1: shows a schematic illustration of a vehicle that has a first embodiment of the device according to the invention;

FIG. 2: shows graphs illustrating the correlation between an isoprene concentration profile and various sleeping and waking states;

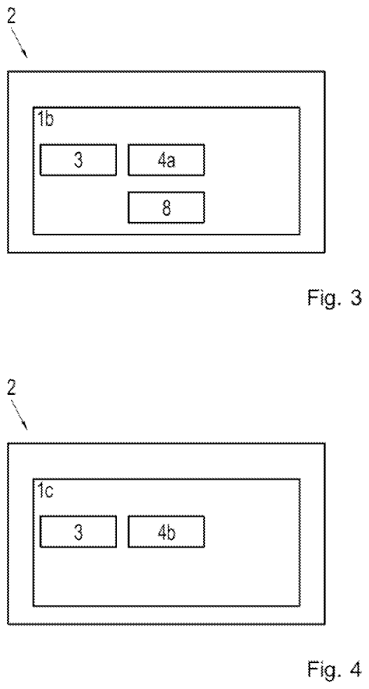

FIG. 3: shows a schematic illustration of a vehicle that has a second embodiment of the device according to the invention;

FIG. 4 shows a schematic illustration of a vehicle that has a third embodiment of the device according to the invention;

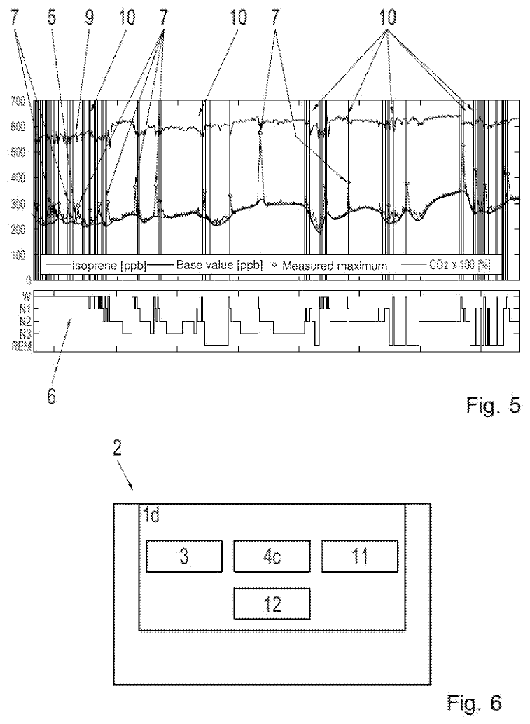

FIG. 5 shows graphs illustrating the correlation between an isoprene concentration profile, a CO2 profile and the various sleeping and waking states;

FIG. 6: shows a schematic illustration of a vehicle that has a fourth embodiment of the device according to the invention;

FIG. 1 shows a device 1a for determining a driver's alertness in a vehicle 2. The device 1a comprises at least one gas sensor 3. The gas sensor 3 is designed to detect isoprene in a driver's exhalations and/or emitted from a driver's skin.

The gas sensor 3 has a metal oxide semiconductor base, and can continuously monitor exhalations, in particular the biomarker isoprene in these exhalations. The gas sensor 3 is located in the steering wheel or somewhere in the proximity of the driver's seat. This facilitates monitoring exhalations. It can also be placed on the seatbelt. By placing it in the steering wheel or on the seatbelt, the detection is concentrated on the driver's exhalations.

The device 1a also contains an analyzer 4a. This can be integrated in the gas sensor 3. The analyzer 4a can extract and quantify an isoprene concentration in the range of a few ppt (parts per trillion=1 in 1012 particles) in realtime, e.g. using chemical analysis technologies. Other quick analysis technologies can also be used.

The isoprene concentration in a predefined time period is determined to obtain an isoprene concentration profile 5 (FIG. 2). This takes place in numerous successive time periods, without breaks between them.

FIG. 2 shows an isoprene concentration profile 5 in correlation with the associated waking and sleeping phase graph below it. The NREM (non-REM) sleep phases are broken down into N1, N2, and N3. N1 represents the sleep phase at the transition from the waking phase to the sleeping phase, N2 represents the stable sleeping phase, and N3 represents deep sleep.

These stages N1, N2, N3 display different characteristics in the electrical activity of the brain that can be measured. Body temperature and blood pressure drop, for example during the NREM phase.

REM sleep (rapid eye movement) is characterized by rapid eye movement behind the eyelids. The nervous system is particularly active during REM sleep. It is accompanied by low muscle tone throughout the body.

The waking phase W is also indicated.

As the isoprene concentration profile 5 indicates, the isoprene concentration fluctuates significantly during the waking phase, i.e. there are numerous peaks, unlike in the sleeping phase, where it remains fairly constant. This is because isoprene is flushed out of the muscles, even with minimal contractions in the skeletal muscles (vertical lines 10). The peaks 7 are obtained when a concentration threshold level is exceeded, and can be determined by calculating a standard deviation.

A peak is identified when the concentration exceeds a predefined threshold value, such that it is higher than the standard deviation of the mean isoprene concentration.

Inactivity detection (fatigue/alertness) in the driver can also be based on random distributions (scatterings) of the isoprene concentration.

It is irrelevant whether the isoprene concentration differs over the course of the day when calculating the distribution. Different foods or illnesses can also affect the absolute isoprene concentrations. By calculating a distribution, the fatigue and alertness of a driver can be detected in a simple manner, independently of the time of day, food intake, or health of the driver.

A frequency analysis can also be used to evaluate the isoprene concentration and determine a driver's alertness.

If the device 1a detects fatigue or levels of alertness lying below a first predefined alertness threshold level, a signal can be sent to a control unit that then generates a visible, and/or tactile, and/or audible signal. The visible signal can be formed by a light. A tactile signal can involve vibrations in the driver's seat or steering wheel. An audible signal can be a warning sound.

The first alertness level can be coupled to a first time period. If too few peaks are detected during this predefined time period, or erratic behavior is detected, it can be assumed that the driver is tired or nodding off.

If the device 1a detects fatigue or low levels of alertness lying below a second predefined alertness level, the device 1a can generate control signals for evaluation in an advanced driver assistance system, or instruct a control unit to generate these control signals.

The second alertness level can be coupled to a second time period that is longer than the first time period. If too few peaks are detected during this predefined time period, or erratic behavior is detected, it can be assumed that the driver is tired or nodding off. This also means that the measures taken in the first time period were ineffective.

These commands can be sent directly or indirectly, via the control unit, to the advanced driver assistance system (e.g. a lane keeping assist), emergency assistance system (e.g. emergency braking), or an emergency response system (e.g. eCall) in the vehicle, which then initiates further necessary measures for preventing life-threatening situations.

The second alertness level can be identical to the first alertness level, or it can be lower, and only distinguish the first time period from the second time period. The second alertness level can also differ from the first alertness level.

FIG. 3 shows another embodiment of the device 1b according to the invention for determining a driver's alertness in the vehicle 2. The device 1b comprises at least the first gas sensor 3 and a second gas sensor 8. The first gas sensor 3 is placed such that it mainly detects the exhalations of the driver. The second gas sensor 8 is placed such that it can detect trace gases (VOCs) emitted through the skin. The two gas sensors 3, 8 can be identical. The first gas sensor 3 can be integrated in the steering wheel, and the second gas sensor 8 can be placed where it is near the driver's skin, e.g. in the seat back or headrest.

The analyzer 4a is configured to determine the isoprene concentration from the detected VOCs. The concentration of isoprene in the driver can be determined more precisely from the detection of isoprene emitted through the skin and in the exhalations.

If the device 1b identifies signs of fatigue indicating a level of alertness below the first predefined alertness level, it can then send a signal to a control unit that generates a visible, and/or tactile, and/or audible signal.

If the device 1b identifies signs of fatigue indicating a level of alertness below the second predefined alertness level, the device 1b can generate control signals for evaluation in an advanced driver assistance system, or instruct a control unit to generate these control signals.

FIG. 4 shows another embodiment of the device 1c according to the invention for determining a driver's alertness in a vehicle 2.

The device 1c comprises at least one gas sensor 3. The gas sensor 3 is configured to detect a driver's exhalations, or emissions through the driver's skin. The device 1c according to the invention also contains an analyzer 4b that is configured to extract the isoprene concentration and another biomarker such as the CO2 concentration from the gases and establish a relationship between them.

FIG. 5 shows such an isoprene concentration profile 5 correlated with the associated waking and sleeping phases diagram 6, and correlated with a CO2 concentration profile forming an overall profile, recorded in the same time period. NREM sleep (non-REM sleep) is again subdivided into N1, N2, N3. N1 represents the sleep phase during the transition from waking to sleeping, N2 represent stable sleep, and N3 represents deep sleep. These sleeping phases N1, N2, N3 exhibit characteristics in the electrical activity of the brain that can be measured. REM sleep (rapid eye movement sleep) is characterized by rapid eye movement behind the eyelids. The nervous system is particularly active during REM sleep. It is accompanied by low muscle tone throughout the body.

The waking phase W is also indicated.

As shown by the isoprene concentration profile 5, the isoprene concentration in exhalations fluctuates significantly during the waking phase, i.e. there are numerous peaks, unlike during the sleeping phases, where it remains fairly constant. This is because isoprene is flushed out of the muscles, even with minimal contractions of the skeletal muscles 10 (vertical lines).

As can also be seen in FIG. 5, the CO2 concentration also tends to increase slightly during the sleeping phases. This is mainly because the driver's breathing becomes slower and steadier, such that there is less dilution with inhaled air in the airways.

A detected lack of alertness, or fatigue, identified with the isoprene concentration profile, can thus be checked or confirmed with a detected lack of alertness or fatigue, identified with the CO2 concentration profile 9. This allows for a more precise determination of the alertness, or a confirmation of a determination of the level of alertness.

FIG. 6 shows a fourth embodiment of the device 1d according to the invention for determining a driver's alertness in the vehicle 2.

The device 1d comprises at least the one gas sensor 3. The gas sensor 3 is configured to detect driver's exhalations or emissions through the skin of the driver. There is also an analyzer 4c and a sensor 11 for identifying the driver. The analyzer 4c is configured to adjust the time period and/or a concentration threshold level to the identified driver. This means that the device 1d can be tailored to the current driver, thus increasing the quality of the alertness determination.

This analyzer 4c can also make these adjustments using a machine learning process in an artificial neural network. The training data can be generated the first time that a driver takes a drive.

This takes into account that the isoprene concentration in the driver's exhalations or emitted through the driver's skin can vary substantially, and may change over the course of the day, or with changes in the driver's health.

As a result, absolute as well as relative and binary (presence or absence of trace gases) changes in the respective isoprene concentrations in comparison with an output or nominal value for a specific driver can be incorporated in the determination of the alertness, thus increasing the quality of the determination.

The device 1d also contains an additional sensor 12 for detecting alertness. This additional sensor 12 can be an interior camera for detecting a driver's eyelid movements and/or pupil dilations, and/or head positions and/or yawning frequency, or it can be a sensor for monitoring pulse rates and/or breathing rates. There can also be numerous additional sensors. The alertness detected with the additional sensor 12 and the alertness determined by the number of peaks 7 and/or on the basis of the frequency analysis and/or on the basis of the random distribution (scattering) are combined to obtain an overall alertness level. A more precise and/or quicker determination of a driver's alertness can be obtained through this combination.

### REFERENCE SYMBOLS

1a, 1b, 1c, 1d device
2 vehicle
3 gas sensor
4a, 4b, 4c analyzer
5 isoprene concentration profile
6 waking/sleeping phase diagram
7 peaks
8 second gas sensor
9 CO₂ concentration profile
10 skeletal muscle contractions
11 driver identification sensor
12 additional sensor

1. A device for determining a driver's alertness in a vehicle, comprising:
at least one gas sensor configured to continuously monitor at least a portion of a driver's exhalations and/or volatile organic compounds emitted from the skin of the driver; and
an analyzer configured to:
extract at least one volatile biomarker in the exhalations and/or the emitted volatile organic compounds, wherein the biomarker comprises isoprene (2-methyl-1,3-butadiene;
determine an isoprene concentration over a predefined time period to obtain an isoprene concentration profile;
determine peaks in the isoprene concentration profile; and
determining an alertness of the driver a basis of at least one of:
a number of peaks within the predefined time period,
a frequency analysis of isoprene concentrations within the predefined time period, and/or
random distributions (scattering) of isoprene concentrations within the predefined time period.

2. The device according to claim 1, comprising:
a sensor configured to identify the driver,
wherein the analyzer is configured to adjust at least one of the time period, the determination of the driver's alertness, and/or the driver's identification, in accordance with the identified driver.

3. The device according to claim 2, wherein the analyzer, is configured to adjust the time period, the determination of the driver's alertness, and/or the driver identification, through a machine learning process.

4. The device according to claim 1, comprising:
at least one additional sensor configured to detect the driver's alertness, wherein the device is also configured to combine the alertness detected by the at least one additional sensor with the determined alertness to obtain an overall alertness level.

5. The device according to claim 4, wherein the at least one additional sensor comprises an interior camera for detecting driver's eyelid movements, and/or pupil dilations, and/or head positions, and/or yawning frequencies.

6. The device according to claim 4, wherein the at least one additional sensor comprises a sensor for monitoring a pulse rate and/or for monitoring a breathing frequency.

7. The device according to claim 1, wherein the at least one gas sensor is located in a driver's headrest and/or seat belt, and/or in the steering wheel, and/or the back of the driver's seat, and/or in the ceiling above the driver's seat.

8. The device according to claim 1, wherein the device is configured to generate visible, and/or tactile, and/or audible signals when the driver's alertness falls below a first predefined alertness level.

9. The device according to claim 1, wherein the device is configured to generate control signals for evaluation in an advanced driver assistance system, if the driver's alertness falls below a second predefined alertness level.

10. The device according to claim 1, comprising a second gas sensor configured to continuously detect volatile organic compounds emitted from the driver's skin.

11. The device according to claim 1, wherein the analyzer is configured to:
extract a second volatile biomarker;
determine the concentration of isoprene and the second volatile biomarker over the time period to obtain an overall concentration profile; and
determine the driver's alertness on the basis of the overall concentration profile.

12. A method for determining a driver's alertness, comprising:
continuously detecting at least a portion of a driver's exhalations and/or the volatile organic compounds emitted from a driver's skin using at least one gas sensor;
extracting at least one volatile biomarker in the exhalation and/or the emitted volatile organic compounds by an analyzer, wherein this biomarker is isoprene (2-methyl-1,3-butadiene);
determining an isoprene concentration over a predefined time period to obtain an isoprene concentration profile;
determining peaks in the isoprene concentration profile; and
determining the driver's alertness on a basis of at least one of:
a number of peaks,
a frequency analysis of isoprene concentrations within the predefined time period, and/or
random distributions (scatterings) of isoprene concentrations within the predefined time period.
